(19) United States
(12) Patent Application Publication
(10) Pub. No.: US 2022/0023086 A1
(43) Pub. Date: Jan. 27, 2022

(54) ORTHOPAEDIC BELT WITH GRIPPING ELEMENTS

(71) Applicant: THUASNE, Levallois Perret (FR)

(72) Inventors: Romain PANNETIER, Lyon (FR); Grégoire Boris BERNOUX, Saint-Etienne (FR); Marie-Thérèse VOLDOIRE, Saint-Etienne (FR)

(21) Appl. No.: 17/311,043

(22) PCT Filed: Dec. 4, 2019
(86) PCT No.: PCT/EP2019/083668

§ 371 Date: Jun. 4, 2021

(51) Int. Cl. A61F 5/02

(52) U.S. Cl. CPC A61F 5/028

(57) ABSTRACT

Disclosed is an orthopedic belt for a user, including a first side panel and a second side panel designed to jointly surround at least one portion of the user's body. The first side panel and the second side panel each have a distal end. The belt defines a central line. The first and second side panels are asymmetric with respect to the central line, such that the distal ends are configured to be placed asymmetrically around the user. The first side panel and the second side panel are each provided with a gripping element for the user to position the belt around himself/herself. The gripping elements are arranged symmetrically on the belt with respect to the central line.

The present invention relates to an orthopedic belt for a user, comprising a first side panel and a second side panel, the first side panel and the second side panel each having a dorsal end and a distal end, the first and second side panels being designed to jointly surround at least one portion of the user's body, the belt defining a central line adapted to be placed against the user's spine, the central line defining a primary direction, the dorsal ends of the first and second side panels being connected to each other, the distal ends of the first and second side panels being adapted to be attached reversibly to each other.

In particular, an orthopedic belt provides support or assistance to the spine as needed by the user.

The geometry of the orthopedic belt is then adapted to said needs to provide the desired effort or support.

However, the shape of the belt is likely to make it difficult to place the belt around the user without external assistance. In particular, for an asymmetrical belt, placing the rear of the belt relative to a user's spine is likely to be done improperly.

Thus, such an orthopedic belt is likely to be worn improperly and thus not have the intended utility, or even be harmful to the user.

An objective of the invention is therefore to provide an orthopedic belt that is suitable for providing support to a user and that is easy for a user to put on.

To this end, the purpose of the invention to provide a belt of the aforementioned type, wherein the first and second side panels are asymmetrical with respect to the central line, such that the distal ends are configured to be placed asymmetrically around the user, the first side panel and the second side panel each being provided with a gripping element for the user to position the belt around the user, the gripping elements being symmetrically arranged on the belt with respect to the central line.

The presence of gripping elements placed symmetrically allows for a symmetrical grip even though the orthopedic belt is generally asymmetrical in shape. This then allows the user to correctly picture the placement of the belt and thus position it correctly.

The orthopedic belt may further have one or more of the following features, considered individually or in any technically possible combination:
the ratio of the average distance between the central line and each distal end to the distance between the central line and each gripping element is between 1.5 and 1.75,
each gripping element comprises a textile strip extending between a first end and a second end, the textile strip being linked to the respective first or second side panel at the first and second ends,
the textile strip is free of the respective first or second side panel outside of the first and second ends of the textile strip
the first and second ends of the textile strip are substantially aligned in the primary direction,
the first and second ends of the textile strip extend along respective axes not parallel to each other, the dimension of the textile strip in the primary direction decreasing as it approaches the distal end of the respective first or second side panel,
the belt has a tightening mechanism, comprising:
   an upper row of cable guides on each of the dorsal ends of the first and second side panels,
   a lower row of cable guides on each of the dorsal ends of the first and second side panels,
   a cable threaded through the cable guides of the upper and lower rows of the first side panel and the second side panel, the cable being threaded alternately through the cable guides of the first side panel and the second side panel,
   first and second handle elements,
the cable being connected to the first and second handle elements so that the dorsal ends of the first and second side panels are brought together by tightening the cable around the cable guides of the upper and lower rows when at least one of the first and second handle elements is pulled,
the cable ends being at least substantially fixed to the first handle element, the cable being slidably threaded through the second handle element,
the upper row of cable guides of the first side panel is spaced from the lower row of cable guides of the first side panel by a space, and the upper row of cable guides of the second side panel is spaced from the lower row of cable guides of the second side panel by a space, and/or
the cable is single.

The invention further relates to a method of placing an orthopedic belt as defined above, comprising the following steps:
a user grasping the belt by the gripping elements,
placing the belt around a portion of the user's body by placing the gripping elements symmetrically on either side of said portion of the body, and
attaching the belt around the user's body by fastening the distal ends to each other.

Further features and advantages of the invention will become apparent from the following description of embodiments of the invention, given by way of example only and with reference to the drawings in which:

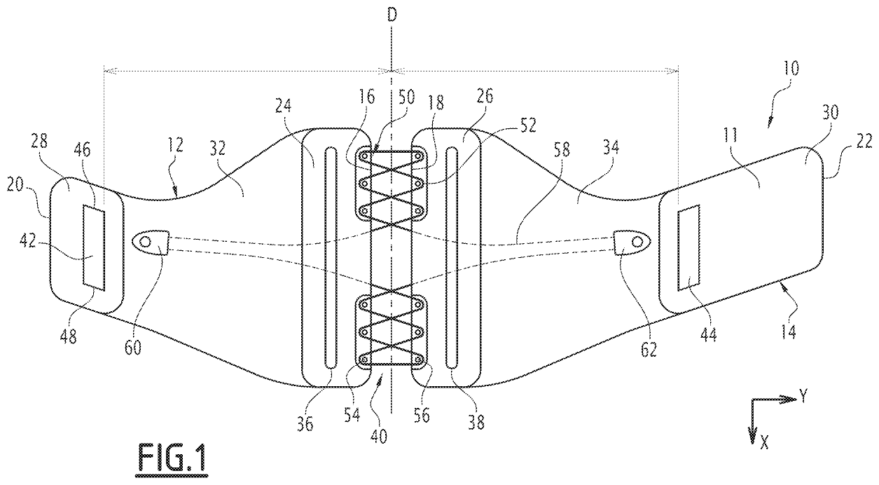

FIG. 1 is a schematic view of a belt according to one embodiment of the invention,

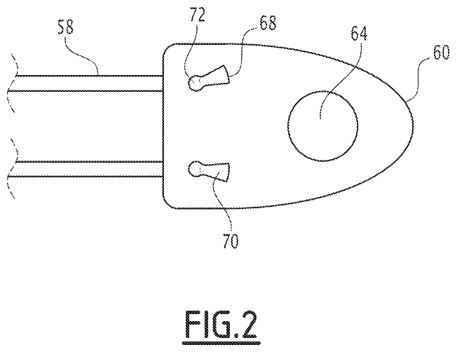

FIG. 2 is a schematic view of a first handle element of the belt of FIG. 1, and

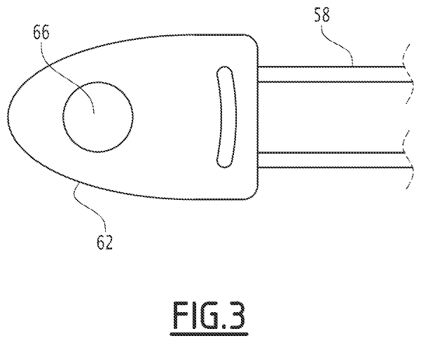

FIG. 3 is a schematic view of a second handle element of the belt of FIG. 1.

An orthopedic belt 10 for a user according to one embodiment of the invention is shown in FIG. 1.

The belt is intended to surround a user's torso when the belt 10 is worn by that user.

The belt 10 is intended to apply pressure on a user when it surrounds said user's torso, in order to support the user's torso, in particular to prevent or relieve back pain.

The belt 10 defines a central line D adapted to be placed against the user's spine.

The central line D extends along a primary direction X.

The belt 10 has an inner face, not visible in FIG. 1, intended to be oriented toward the user's torso and an outer side 11 opposite the inner face, visible in FIG. 1.

The belt 10 comprises a first side panel 12 and a second side panel 14.

The first and second side panels 12, 14 are intended together to surround at least a portion of the user's body, more particularly the torso.

The first and second side panels 12, 14 are asymmetrical to each other with respect to the central line D.

More particularly, one of the side panels 14 has a dimension along a direction perpendicular to the primary direction X, known as the extension direction Y, that is strictly greater than that of the other of the side panels 12.

The ratio of the dimension of one of the side panels 14 along the extension direction Y, to the dimension of the other of the side panels 12 along the extension direction Y, is greater than or equal to 1.1, for example.

In particular, this allows for a large adjustment range, in particular to adapt the belt to a user's body measurements, and/or a large tightening range, depending on the effort required to relieve the user.

Each side panel 12, 14 has a dorsal end 16, 18 and a distal end 20, 22 opposite the dorsal end 16, 18. The dorsal end 16, 18 and the distal end 20, 22 are opposite each other along the extension direction Y.

The dorsal ends 16, 18 are arranged on either side of the central line D, and thus intended to be placed on either side of the spine, more particularly near a user's spine when the belt is worn. The dorsal ends 16, 18 are intended to be placed symmetrically with respect to each other with respect to said spine.

The dorsal ends 16, 18 of the first and second side panels 12, 14 and thus the side panels 12, 14 are further connected to each other via a tightening mechanism 40, for example, which will be described later in more detail.

Alternatively, the dorsal ends 16, 18 are connected by another attachment system or are integral so that the first side panel and the second side panel form a single piece.

The distal ends 20, 22 of the first and second side panels 12, 14 are adapted to be attached reversibly to each other.

Because the side panels 12, 14 are asymmetrical with respect to the central line D, the distal ends 20, 22 are configured to be placed asymmetrically around the user, more particularly on the user's belly.

More particularly, each side panel 12, 14, comprises a dorsal portion 24, 26, intended to extend beside a user's spine when the belt 10 is worn, a distal portion 28, 30, and an intermediate portion 32, 34, between the dorsal portion 24, 26, and the distal portion 28, 30.

The dorsal portion 24, 26, the intermediate portion 32, 34, and the dorsal portion 24, 26, of a side panel 12, 14 are aligned along the extension direction Y.

Each dorsal portion 24, 26 defines the corresponding dorsal end 16, 18 and each distal portion 28, 30 defines the corresponding distal end 20, 22.

The dorsal portions 24, 26, are substantially symmetrical to each other with respect to the central line D.

The dorsal portions 24, 26 are not elastic, that is, they cannot be stretched.

Each dorsal portion 24, 26 comprises at least one retaining element 36, 38.

The retaining elements 36, 38 of the dorsal portions 24, 26 of the side panels are positioned symmetrically on either side of the central line D.

In the embodiment shown, each retaining element 36, 38 is a wire, here extending substantially parallel to the corresponding dorsal end 16, 18.

Alternatively, each dorsal portion 24, 26 comprises two retaining elements, here wires, the retaining elements of the dorsal portions extending symmetrically on either side of the central line D. Each dorsal portion 24, 26 comprises a first retaining element proximate to and extending substantially parallel to the corresponding dorsal end 16, 18, for example, and a second retaining element, further from the dorsal end 16, 18 than the first retaining element. The second retaining element here is not parallel to the dorsal end.

The intermediate portions 32, 34 are substantially symmetrical to each other with respect to the central line D.

The intermediate portions 32, 34 are elastic. They are woven or knitted from elastic threads, for example.

“Elastic” is understood here as the belt 10 in said portions being capable of being stretched by at least 10% without damage and, in the absence of stretching, capable of returning to its original dimensions.

The distal portions 28, 30 are asymmetrical to each other with respect to the central line D. More particularly, one of the distal portions 30 has a dimension along the extension direction Y that is strictly greater than that of the other of the distal portions 28.

The ratio between the dimension along the extension direction Y of one of the distal portions 30 and that of the other of the distal portions 28 is strictly greater than 1.25 here, more particularly greater than or equal to 1.50. Said ratio depends on the adjustment range, for example, and/or the dimensions of the reversible fastening of distal ends described below.

Here, the distal portions 28, 30 are not elastic, that is, they cannot be stretched.

To enable the reversible fastening of the distal ends to each other, the distal portions 28, 30 are each provided with at least one complementary loop or hook type of self-adhesive strip at one end on the inner side and at the other end on the outer side, for example.

More particularly, the outer side of the distal portion 28, 30 of at least one of the side panels 12, 14, more particularly the first and second side panels, is formed with one of the loop or hook types.

One of the side panels is provided with a portion of the other of the loop or hook types on its inner side.

The hook and loop strips interact with each other when the belt is closed around the user's torso.

The first side panel 12 and the second side panel 14, more particularly the distal portions 28, 30, are further each provided with a gripping element 42, 44 for the user to position the belt around the user.

The gripping elements 42, 44 are arranged symmetrically on the belt 10 with respect to the central line D.

The ratio between the average distance between the central line D and each distal end 20, 22 and the distance between the central line D and each gripping element 42, 44 is here between 1.5 and 1.75.

The ratio between the average distance between the central line D and the distal end 20 of the first side panel 12 and the distance between the central line D and the corresponding gripping element 42 is here greater than or equal to 1.

The ratio between the average distance between the central line D and the distal end 22 of the second side panel 14 and the distance between the central line D and the corresponding gripping element 44 is here less than or equal to 2.

Each gripping element 42, 44 comprises a textile strip extending between a first end 46 and a second end 48.

The textile strip is configured so that a user is able to slide a hand or finger, such as a thumb, between the textile strip and the outer side of the corresponding side panel.

The textile strip is attached to the respective first or second side panel 12, 14 at the first and second ends 46, 48.

Here, the textile strip is stitched to the distal portion 28, 30 of the corresponding side panel 12, 14.

The textile strip has an outer side, advantageously formed of the same type of loop or hook as the outer side 11 of the corresponding side panel 12, 14.

Here, the textile strip is a quadrilateral with two opposite sides forming the first and second ends 46, 48.

The textile strip is free of the respective first or second side panel 12, 14 outside of the first and second ends 46, 48 of the textile strip.

The first and second ends 46, 48 of the textile strip are substantially aligned along the primary direction.

The first and second ends 46, 48 of the textile strip extend along respective axes that are not parallel to each other.

The dimension of the textile strip along the primary direction decreases as it approaches the distal end 20, 22 of the respective first or second side panel 12, 14.

The dimension of the textile strip along the primary direction at its end closest to the dorsal end 16, 18 is between 3 cm and 15 cm, for example, for the passage of a finger or a hand, more particularly between 8 cm and 15 cm, especially for the passage of a hand.

The dimension of the textile strip along the primary direction at its end closest to the distal end 20, 22 is between 2 cm and 10 cm, for example, for the passage of a finger or a hand, more particularly between 5 cm and 10 cm, especially for the passage of a hand.

In particular, this facilitates having dimensions particularly suitable for a user's hand to slip through.

The previously mentioned tightening mechanism 40 is now described in more detail.

The tightening mechanism 40 comprises cable guides 50, 52, 54, 56, a cable 58, more particularly a single cable, a first handle element 60 and a second handle element 62.

More particularly, the tightening mechanism 40 comprises an upper row of cable guides 50, 52 and a lower row of cable guides 54, 56, on each of the dorsal portions 24, 26 of the first and second side panels 12, 14.

The upper row of cable guides 50 of the first side panel 12 is spaced from the lower row of cable guides 54 of the first side panel 12 by a space. The space is greater than 2 millimeters (mm). They are substantially aligned along the primary direction.

Advantageously, the space is greater than the distance between two adjacent guides in each of the rows of guides 50, 54, more particularly greater than 35 millimeters here.

The upper row of cable guides 52 of the second side panel 14 is spaced from the lower row of cable guides 56 of the second side panel 14 by a space. The space is greater than 2 millimeters (mm). They are substantially aligned along the primary direction.

Advantageously, the space is greater than the distance between two adjacent guides in each row of guides 52, 56, more particularly greater than 35 millimeters here.

Each dorsal portion 24, 26 is provided with a upper plate and a lower plate, for example, the upper row of cable guides 50, 52 being arranged on the upper plate and the lower row of cable guides 54, 56 being arranged on the lower plate.

The plates of a single side panel 12, 14 are spaced at a distance of between 1 millimeter (mm) and 20 centimeters (cm).

Each cable guide is made in one piece with the corresponding plate, for example, thus reducing the number of components of the tightening mechanism. According to another embodiment, the cable guides are attachments, fixed to the plate, for example.

Here, each cable guide delimits a passage for guiding the cable.

Each cable guide 50, 52, 54, 56 comprises a fixed lug, for example, around which the cable is intended to be threaded.

The fixed lug extends from the corresponding plate.

The lug is cylindrical and extends along an axis substantially perpendicular to the plane in which the plate extends so that a cable, arriving at the lug in an initial direction, passes around the lug and exits the lug in a direction forming a non-zero angle with the initial direction.

Each cable guide further comprises a means for retaining the cable around the pin. The retaining means is a groove, a tube, or a upper and/or radial stop, for example.

Alternatively, each cable guide is formed by a pulley.

The cable 58 comprises a first end 68 and a second end 70.

The cable 58 is threaded through the cable guides of the upper and lower rows of the first side panel 12 and the second side panel 14. More particularly, the cable 58 is threaded alternately through the cable guides of the first side panel 12 and the second side panel 14, more particularly through the upper guide rows 50, 52 on the one hand and the lower guide rows 54, 56 on the other hand.

This forms a cross-lacing of the cable 58 between the first side panel 12 and the second side panel 14, more particularly in a lower portion on the one hand and an upper portion of the belt on the other hand.

The cable 58 is connected to the first and second handle elements 60, 62, so that the dorsal ends 16, 18 of the first and second side panels 12, 14 are brought together by tightening the cable 58 around the cable guides of the upper and lower rows 50, 52, 54, 56 when one of the handle elements 60, 62 is pulled.

Each handle element 60, 62 comprises at least one opening 64, 66, for example, to allow gripping of said handle element 60, 62 to allow a user to pull on the handle element 60, 62.

The first and second ends of the cable 68, 70 are fixedly attached to the first handle element 60, with the cable 58 slidably threaded through the second handle element 62.

More particularly, each handle element 60, 62 has two through openings 72.

The cable 58 has an enlargement at the first end 68 and the second end 70, intended to keep each end 68, 70 stationary at a respective through opening 72.

This is achieved, for example, by knotting said cable at the ends 68, 70 after passing each end through a respective through opening of the first handle element 60.

Alternatively, the ends 68, 70 of the cable 58 are attached together after each end of the cable has passed through a respective through opening 72. The cable ends are then slidable relative to the first handle element 60 by a distance less than the distance between the two through openings. It is considered here that the cable ends 68, 70 are then substantially fixed relative to the first handle element 60.

Moreover, the cable 58 passes successively through the two through openings of the second handle element 62. This allows the cable to slide through the two through openings.

This sliding of the cable relative to the second handle element 62, in particular, allows for adjusting the ratio between the length of the cable 58 in one upper portion of the belt 10 and the length of the cable 58 in one lower portion the belt 10, so as to adjust the belt 10 better to the user's morphology.

Indeed, by increasing the length of cable 58 in an upper portion, this spreads the dorsal ends 16, 18 in the upper portion of the belt that much further apart relative to the lower portion, so that it is particularly suitable for a V-shaped body shape. Conversely, by decreasing the length of cable 58 in an upper portion, this brings the dorsal ends 16, 18 in the upper portion of the belt 10 that much closer relative to the lower portion, so that this is particularly suitable for a \hat{ } morphology.

A method for placing an orthopedic belt according to the invention will now be described.

A user grasps the belt 10 using the gripping elements 42, 44, in particular by passing each of the user's hands through one of the gripping elements, more particularly between the belt and the textile strip of the gripping element.

The user places the belt 10 around his/her torso with the central line D against the spine. This is easily accomplished by placing the gripping elements on either side of his/her torso symmetrically between his/her right and left sides.

Once the belt is properly positioned, the user attaches the belt 10 by securing the distal ends 20, 22 to each other.

The presence of the gripping elements thus provides a symmetrical grip for the user, who then has a marker for correctly placing the central line of the belt against his/her spine, without the need for external assistance.

Correct placement of the belt ensures that the belt has the desired effect on the user.

1. An orthopedic belt for a user, comprising a first side panel and a second side panel, each of the first side panel and the second side panel having a dorsal end and a distal end, the first and second side panels being designed to jointly surround at least one portion of the user's body, the belt defining a central line adapted to be placed against the user's spine, the central line defining a primary direction,
the dorsal ends of the first and second side panels being connected to each other,
the distal ends of the first and second side panels being adapted to be reversibly attached to each other,
the first and second side panels being asymmetrical with respect to the central line, such that the distal ends are configured to be placed asymmetrically around the user,
the first side panel and the second side panel each being provided with a user gripping element for positioning the belt around the user,
the gripping elements being arranged symmetrically on the belt with respect to the central line.

2. The orthopedic belt according to claim 1, wherein the ratio of the average distance between the central line and each distal end to the distance between the central line and each gripping element is between 1.5 and 1.75.

3. The orthopedic belt according to claim 1, wherein each gripping element comprises a textile strip extending between a first end and a second end, the textile strip being linked to the respective first or second side panel at the first and second ends.

4. The orthopedic belt according to claim 3, wherein the textile strip is free of the respective first or second side panel outside of the first and second ends of the textile strip.

5. The orthopedic belt according to claim 3, wherein the first and second ends of the textile strip are substantially aligned along the primary direction.

6. The orthopedic belt according to claim 5, wherein the first and second ends of the textile strip extend along respective axes not parallel to each other, the dimension of the textile strip along the principal direction decreasing as it approaches the distal end of the respective first or second side panel.

7. The orthopedic belt according to claim 1, comprising a tightening mechanism comprising:
an upper row of cable guides on each of the dorsal ends of the first and second side panels,
a lower row of cable guides on each of the dorsal ends of the first and second side panels,
a cable threaded through the upper and lower rows of cable guides of the first side panel and the second side panel, the cable being threaded alternately through the cable guides of the first side panel and the second side panel,
a first and a second handle elements,
the cable being connected to the first and second handle elements such that the dorsal ends of the first and second side panels are brought together by tightening the cable around the upper and lower row cable guides when at least one of the first and second handle elements is pulled,
the ends of the cable being at least substantially fixedly attached to the first handle element, the cable being slidably threaded through the second handle element.

8. The orthopedic belt according to claim 7, wherein the upper row of cable guides of the first side panel is spaced from the lower row of cable guides of the first side panel by a space and the upper row of cable guides of the second side panel is spaced from the lower row of cable guides of the second side panel by a space.

9. The orthopedic belt according to claim 7 or 8, wherein the cable is unique.

10. A method of placing an orthopedic belt according to claim 1, comprising the following steps:
a user grasping the belt by the gripping elements,
placing the belt around a portion of the user's body by placing the gripping elements symmetrically on either side of said portion the body, and
securing the belt around the user's body by fastening the distal ends to each other.

11. The orthopedic belt according to claim 2, wherein each gripping element comprises a textile strip extending between a first end and a second end, the textile strip being linked to the respective first or second side panel at the first and second ends.

12. The orthopedic belt according to claim 4, wherein the first and second ends of the textile strip are substantially aligned along the primary direction.

13. The orthopedic belt according to claim 8, wherein the cable is unique.
